(19) United States
(12) Patent Application Publication
(10) Pub. No.: US 2016/0114128 A1
(43) Pub. Date: Apr. 28, 2016

(54) Actuating Catheter Luer Devices

(71) Applicant: AngioDynamics, Inc., Latham, NY (US)

(72) Inventor: Carol Lancette, Fort Ann, NY (US)

(73) Assignee: ANGIODYNAMICS, INC., Latham, NY (US)

(21) Appl. No.: 14/523,025

(22) Filed: Oct. 24, 2014

(51) Int. Cl. A61M 25/00; A61M 39/24; A61M 39/06; A61M 39/10

(52) U.S. Cl. CPC A61M 25/0075; A61M 25/0069; A61M 39/1011; A61M 25/0097; A61M 39/24; A61M 39/06; A61M 2025/0079; A61M 2025/0078; A61M 2025/0076; A61M 2039/1044; A61M 2039/1038; A61M 2039/2426; A61M 2039/062; A61M 2039/064; A61M 2039/0646

(57) ABSTRACT

A luer for a catheter is disclosed. The luer has a proximal luer housing having a proximal opening, a distal luer housing having a stem and a distal opening, and a threaded element disposed on an external surface of the luer. The proximal housing and the distal housing define a fluid channel, and the proximal housing is slidably attached to the distal housing so that in a closed configuration the proximal opening is fluidly sealed by the stem, and in an open configuration the fluid channel is in fluid communication with the proximal opening. The luer can be formed integral to a catheter hub.

### FIELD OF THE INVENTION

The present invention relates generally to medical devices for establishing fluid access to a target site within a human body. More specifically, the invention relates to cathete with actuating luers, and integrated luer and hub designs.

### BACKGROUND OF THE INVENTION

Medical devices such as catheters are commonly used by medical professionals for establishing fluid access with a target area within the human body. Common types of catheters include dialysis catheter peripherally inserted central catheters, midline catheters, central venous catheters and drainage catheters. The type of catheter used on a particular patient is typically based on a number of factors, including the procedure being performed and the area within the human body that needs to be accessed. One common feature of these types of conventional catheters is that they often have a proximal end configuration that includes a hub, and extension tube, a luer, and a clamp surrounding the extension tube for closing-off fluid access to the catheter lumen between procedures. Closing fluid access to the catheter lumen while the catheter is riot in use is an important safety precaution for minimizing risk of air embolism, exsanguination no contamination.

A conventional dialysis catheter is shown in FIG. 14. The main body of the catheter 300 features an elongate shaft 312 having a proximal end 314 and a distal end 316. The elongate shaft 312 is typically a dual lumen shaft that terminates at the distal end 316 in a step tip (as shown) or split tip configuration. The hub 318 provides a transition and bifurcation point for each of the catheter lumens to split of individually into separated tubing at the proximal end 314 of the device. In addition, the hub 318 often doubles as an anchor point for the catheter, often including one or more attachment elements such as a suture wing for anchoring the device to the body of the patient. Each separated lumen branches out into a respective extension tube 324, 326. The arterial lumen branches out to a first extension tube 324, which is surrounded by a clamp 328. The proximal end of the arterial lumen extension tube 324 terminates in a luer 320. Likewise, the venous lumen branches out to a second extension tube 326, which is surrounded by a clamp 330. The proximal end of the venous lumen extension tube 326 also terminates in a luer 322. The clamps 328, 330 can be closed to seal off the extension tube 324, 326 lumens.

Problems may arise with the use of extension tubes and clamps to seal off fluid access. For instance, frequent opening and closing of the clamps can start to weaken the extension tube, which may ultimately require an extension tube repair, or replacement of the entire catheter. Further, clamps surrounding the extension tube are limited to clamping the extension tube, and do not typically extend to more proximal points on the device, such as the luer. So even when the clamp is closed, the parts of the extension tube lumen and luer interior that are proximal of the clamp remain exposed to fluid and air communication, giving rise to potential contamination. It would be desirable to reduce the proximal end weight and bulk of the catheter, while reducing production costs associated with manufacturing the catheter. Still further, it would be desirable to fluidly seal lumens on the device at point more proximal than conventional extension tube and clamp configurations. In addition, it would be desirable to close off proximal end fluid access to lumens, without requiring a separate accessory component such as an end cap or other accessory device.

### SUMMARY OF THE INVENTION

According to certain embodiments, the invention is a luer for a catheter. The luer has a proximal luer housing having a proximal opening, a distal luer housing having a stern and a distal opening, and a threaded element disposed on an external surface of the luer. The proximal housing and the distal housing define a fluid channel, and the proximal housing is slidably attached to the distal housing so that in a closed configuration the proximal opening is fluidly sealed by the stem, and in an open configuration the fluid channel is in fluid communication with the proximal opening.

According to another embodiment, the invention is a catheter having an elongate flexible shaft having a first lumen, and a hub having a first luer. The first luer has a first proximal luer housing comprising a first proximal opening, a first distal luer housing having a first stern and a first distal opening, and a first threaded element disposed on an external surface of the first luer. The first proximal housing and the first distal housing define a first fluid channel, the first proximal housing is slidably attached to the first distal housing so that in a closed configuration the first proximal opening is fluidly sealed by the first stem, and in an open configuration the first proximal opening is in fluid communication with the first fluid channel and the first lumen. The first distal luer housing is formed integral to the hub.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a side view of a dialysis catheter according to on exemplary embodiment of the invention;

FIG. 2 is a side view of a luer in closed position according to are exemplary embodiment of the invention;

FIG. 3 is side view of the luer shown in FIG. 2 in open position;

FIG. 4 is a cross-sectional side view of the luer shown in FIG. 2 in closed position;

FIG. 5 is a cross-sectional side view of the luer shown in FIG. 2 in open position;

FIG. 6 is a cross sectional view of the luer shown in FIG. 2 along cross section 6-6 shown in FIG. 5;

FIG. 7 is a cross-sectional side view of a luer with an offset stem in open position according to an exemplary embodiment of the invention;

FIG. 8 is a cross-sectional view of the luer shown in FIG. 7 along cross section 8-8;

FIG. 9 is a side view of a luer with a guide wire opening in a lower housing and in closed position according to an exemplary embodiment of the invention;

FIG. 10 is a cross-sectional side view of the luer shown in FIG. 9;

FIG. 11 is a side view of a luer with a twisting mechanism according to an exemplary embodiment of the invention;

FIG. 12 is a cutaway view of the valve mechanism inside of the luer shown in FIG. 11 in open position;

FIG. 13 is a cutaway view of the valve mechanism inside of the luer shown in FIG. 11 in closed position; and

FIG. 14 is a side view of a prior art dialysis catheter.

### DETAILED DESCRIPTION OF THE INVENTION

The present invention can be understood more readily by reference to the following detailed description, the examples included therein, and to the Figures and their following description. The drawings, which are not necessarily to scale, depict selected preferred embodiments and are not intended to limit the scope of the invention.

The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

Referring now in detail to the drawings, in which like reference numerals indicate like parts or elements throughout the several views, in various embodiments, presented herein are improved catheter luer and hub devices.

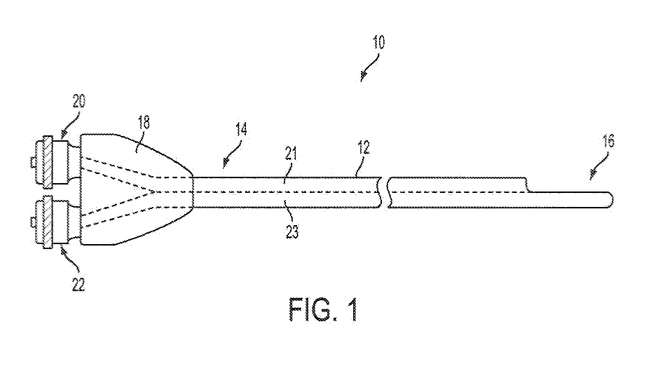

A first exemplary embodiment of the invention is shown in the dialysis catheter of FIG. 1. The dialysis catheter 10 has a flexible elongate shaft 12 that extends from a proximal end 14 to a distal end 16. The elongate shaft 12 has an arterial lumen 21 for removing blood from the body and a venous lumen 23 for returning blood to the body during a dialysis treatment. The distal end 16 of the shaft 12 terminates in a step tip configuration offsetting the distal openings of each lumen 21, 23. The step tip configuration is known in a common configuration for minimizing blood recirculation rates during treatment. The proximal end of the lumens 21, 23 continue through the hub 18, where they bifurcate and extend into fluid communication with a respective luer 20, 22. Each luer can be coded with a label and/or color to indicate whether it is the luer for the arterial lumen or the venous lumen. The luers 20, 22 are integrally formed with the catheter hub 18.

The shaft 12 can be made of a medical grade polymer, such as a medical grade urethane. The shaft may also include a thromboresistant admixture, such as those polymer and fluoropolymer admixtures disclosed in U.S. Pat. No. 8,784,402 and U.S. Patent Pub. No. 2014/0276470, both to Lareau et al., and both of which are incorporated herein by reference. The leers 20, 22 can be integrally formed with or attached to the hub 18 by a number of methods known in the art. For instance, the hub and luers 20, 22 can be attached utilizing a chemical bond, such as cyclohexanone, or an adhesive bond, such as a UV curable adhesive. Injection molding and/or overmolding techniques can also be used to mold the hub 18 and luers 20, 22 as integral components. The hub and luers can also be manufactured using anti-thrombogenic additives as part of an admixture polymer. It will be understood by those having ordinary skill in the art that if desired, the luers described herein can also be utilized with extension tubes and will maintain certain advantages described herein for a clampless catheter. Chemical bonds, adhesive bonds and over-molding techniques described above can be utilized along with techniques known in the art for attaching the luers described herein to extension tubes.

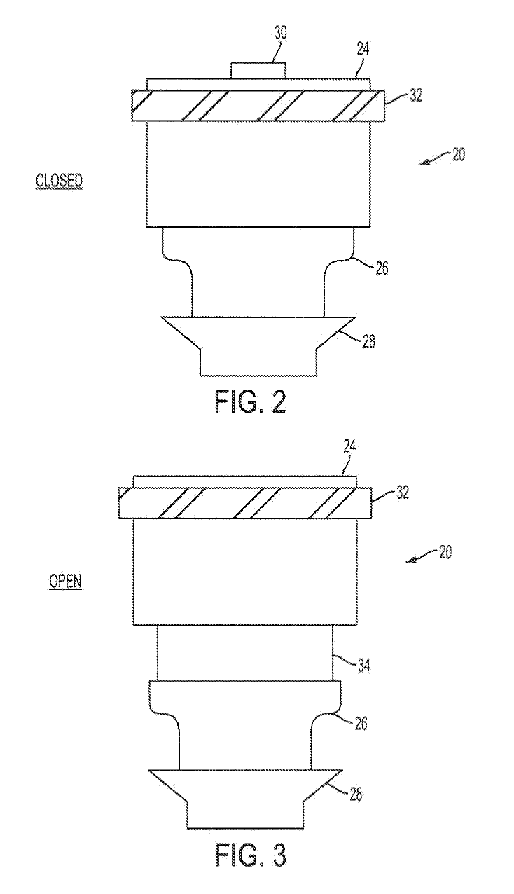

With reference to FIGS. 2 and 3, a luer 20 is shown in closed and open positions respectively according to an exemplary embodiment of the invention. The luer 20 includes a proximal housing 24 and a distal housing 26 that are slidably connected to each other in a mating configuration. The distal housing may include an element such as a barb 28 for facilitating attachment to a hub or an extension tube. The proximal housing 24 has a threaded element 32 so that the device can be attached to tubing connections such as tubing connections for dialysis machine or syringes for flushing the device. The slidable connection between the proximal housing 24 and the distal housing 26 allows medical practitioners to easily slide or “pop” the luer 20 from a closed configuration (shown in FIG. 2) to an open configuration (shown in FIG. 3), using a simple grasping, pulling or pushing motion with one hand.

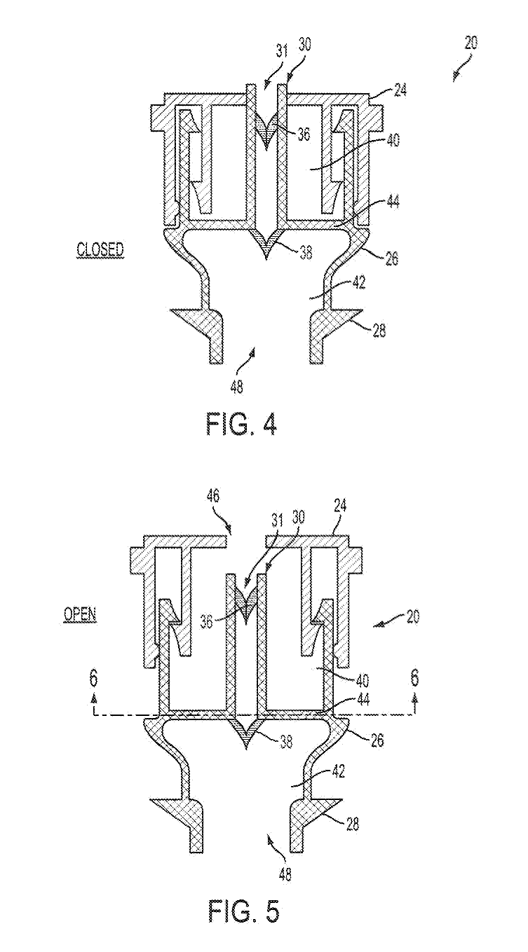

A cross-sectional view of the luer 20 in closed and open positions respectively is shown in FIGS. 4 and 5. The distal housing 26 includes a stem 30 having a stem lumen 31. The stern lumen 31 is fluidly sealed by a pair of elastomeric duckbill valves 36, 38. The valves 36, 38 are biased shut so that they remain closed when subjected to the positive and negative pressure fluctuations of a dialysis procedure, as well as internal body pressure fluctuations. Upon the insertion of a guide wire or a stylet into the stem lumen 31, the valves are configured to open, providing compatibility for wire accessories as needed. In the open position, the proximal opening 46 is in fluid communication with the interior 40 of the proximal housing 24 and the interior 42 of the distal housing 26. A supporting member 44 supports the stem 30, and has openings 43 (as shown in FIG. 6) to allow for fluid communication between the proximal housing 24 interior 40 and the distal housing 28 interior 42. Thus, when in the open configuration, fluid can traverse the interior of the luer housings from the distal opening 48 of the distal housing 26 to the proximal opening 46 of the proximal housing 24. In the closed configuration, fluid cannot traverse to the proximal opening 46 since it is fluidly sealed by the stem 30 protruding through the proximal opening 46. The elastomeric valves 36, 38 can be other types of valves, such as slit valves. A duckbill valve 38 positioned at the distal end of the stem lumen 31 may be especially advantageous since it diverts fluid away from the stem lumen 31 and towards the openings 43. Since fluid communication through the luer can be easily established via slidable actuation by the user, there is no need for extension tubes and clamps, resulting in less proximal end weight, less materials, streamlined manufacturing and a cost savings. Further, patient safety outcomes are improved due to the minimization of extension tube fatigue. In certain embodiments, the luer can be curved up and away from the hub so that when the hub is secured fiat to the skin of the patient's body, the luer connection is more easily accessible to the medical provider. Further, the luer and/or hub can be made from a polymer having translucent properties so that the medical provider can properly verify flashback and fluid flow through the proximal ends of the device lumens.

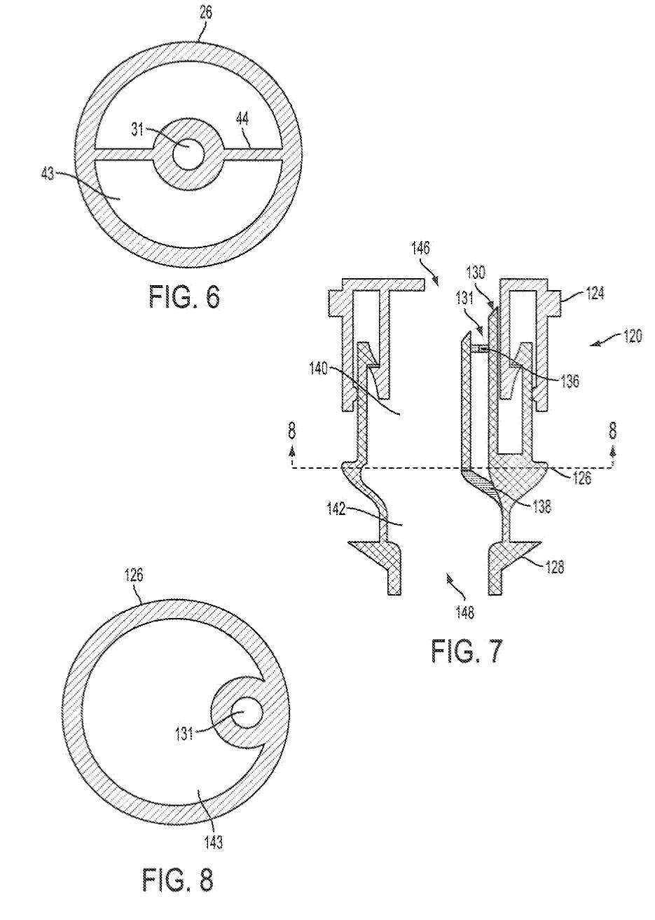

A cross-sectional view of a luer 120 according to an alternative embodiment of the invention is shown in FIG. 7. The luer 120 has a proximal housing 124 and a distal housing 126 that are connected in a slideable mating relationship to each other. The distal housing 126 can have a barb 128 as described in previous embodiments. As shown with additional reference to FIG. 8, the distal housing 126 also includes a stem 130 that is offset from the center of the luer, allowing fluid to flow through a more central opening 143 as it passes through the interior 140 of the luer 120 between the proximal opening 146 and the distal opening 148. The stern 130 can be supported by a bulked-up sidewall to the distal housing 126, further configured to form the stem lumen 131. The offset stem lumen 131 can have a pair of elastorneric valves 136, 138 to fluidly seal the stem lumen 131, while allowing for the traversal of a guide wire or stylet as described in previous embodiments. The top of the stem 130 can also feature angled walls for diverting fluid towards the interior 140 of the luer 120.

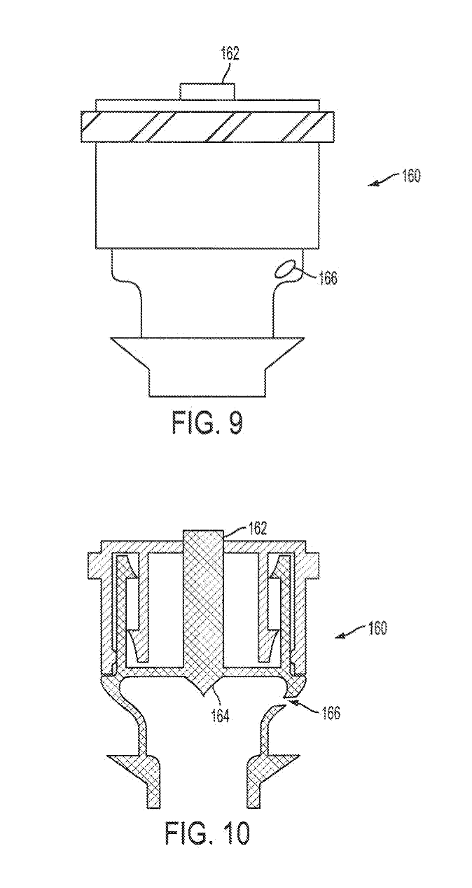

An alternative embodiment of the invention with a modified stem and guide wire access structure is shown in dosed configuration in FIGS. 9 and 10. The luer 160 features a stem 162 that does not contain any fluid lumens. The bottom of the stem 164 is geometrically shaped to divert fluid away from the stern, similar to the valve structure shown in the embodiment of FIGS. 4 and 5. A port 166 is provided to allow for the introduction of a guide wire or stylet. The port 166 can be fluidly sealed by a self-sealing elastomeric member, such as a gasket.

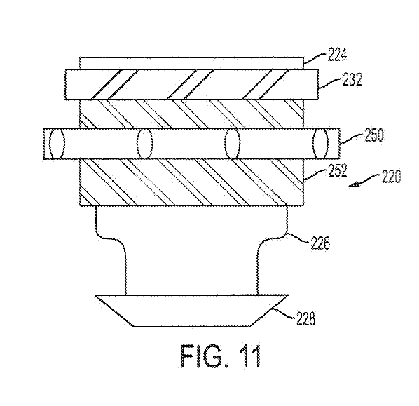

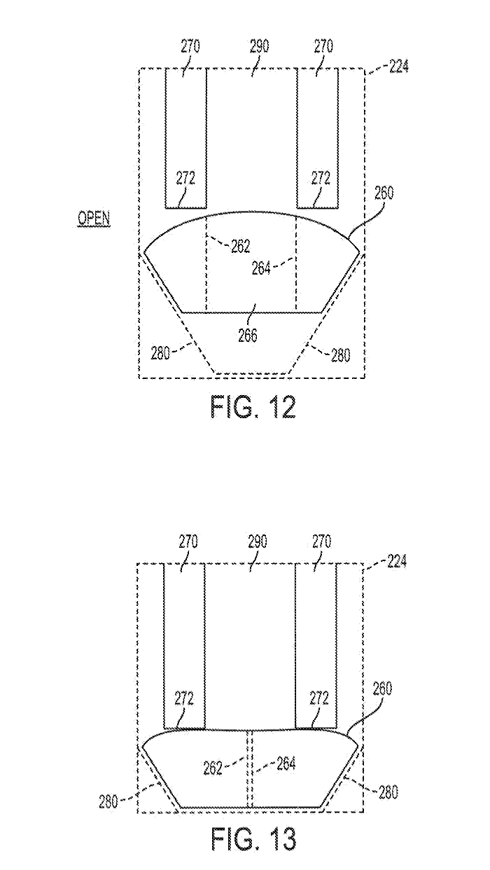

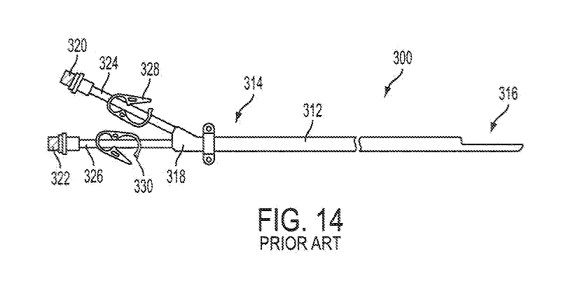

Luers according to certain embodiments of the invention can include a system similar to a tuohy borst valve for closing and opening fluid access through the luer. In the embodiment shown in FIGS. 11-13, a luer 220 includes a proximal housing 224, a distal housing 226 and a barb 228 on the distal housing 226. A tubing threaded element 232 is formed on the luer 220 for attaching external device tubing, such as tubing for a dialysis machine. The luer 220 also includes an actuation threaded element 252 and an actuation collar 250 loaded over the threaded element 252. The actuation collar 250 is configured to raise actuation members 270 in the open position as shown in FIG. 12, and to lower actuation members 270 in the dosed position as shown in FIG. 13. The interior of the luer 220 includes an elastomeric insert 260 that has an open lumen 266 in a relaxed configuration As the actuation collar 250 is twisted towards a closed position the bottom 272 of the actuation members 270 will push the elastomeric insert 260 along funnel shaped walls 280, closing the lumen wails 262, 264 so that the lumen 266 completely blocks off fluid communication. To reestablish fluid communication, the actuation collar 250 can be twisted towards an open position, allowing the elastomeric insert 260 to expand and move back up the funnel shaped walls 280, allowing the lumen 266 to expand.

What is claimed is:

1. A luer for a catheter comprising:
a proximal luer housing comprising a proximal opening;
a distal luer housing comprising a stern and a distal opening; and
a threaded element disposed on an external surface of the luer;
wherein the proximal housing and the distal housing define a fluid channel, and
wherein the proximal housing is slidably attached to the distal housing so that in a closed configuration the proximal opening is fluidly sealed by the stem, and in an open configuration the fluid channel is in fluid communication with the proximal opening.

2. The luer of claim 1, wherein the stem further comprises a stem lumen.

3. The luer of claim 2, wherein the stem lumen is fluidly sealed by a first elastorneric valve.

4. The luer of claim 3, wherein the elastorneric valve is a duckbill valve.

5. The luer of claim 3, wherein the stem lumen is fluidly sealed by a second elastomeric valve.

6. The luer of claim 5, wherein the first elastorneric valve is disposed on the proximal end of the stem lumen, and the second elastomeric valve is disposed on the distal end of the stem lumen.

7. The luer of claim 2, wherein a duckbill valve is disposed distal of the stem lumen and configured to divert fluid away from the stem lumen.

8. The luer of claim 2, wherein the stem lumen is configured to accept a medical guide wire or stylet.

9. The luer of claim 1, wherein the stem is substantially centered within the fluid channel.

10. The luer of claim 1, wherein the stern is substantially off-center from the fluid channel.

11. The luer of claim 10, wherein a wall of the stem are configured to divert fluid towards the proximal opening.

12. The luer of claim 1, wherein a wire opening is disposed in a wall of the distal lumen housing.

13. The luer of claim 12, wherein the wire opening is fluidly sealed by an elastomeric member.

14. The luer of claim 1, wherein a fluid diversion member configured to divert fluid towards the fluid channel is disposed at a distal end of the stern.

15. The luer of claim 1, wherein at least one of the distal luer housing and the proximal luer housing comprises a translucent material.

16. A catheter comprising:
an elongate flexible shaft comprising a first lumen; and
a hub comprising a first luer, the first luer comprising:
a first proximal luer housing comprising a first proximal opening,
a first distal luer housing comprising a first stem and a first distal opening, and
a first threaded element disposed on an external surface of the first luer;
wherein the first proximal housing and the first distal housing define a first fluid channel,
wherein the first proximal housing is slidably attached to the first distal housing so that in a closed configuration the first proximal opening is fluidly sealed by the first stem, and in an open configuration the first proximal opening is in fluid communication with the first fluid channel and the first lumen, and
and wherein the first distal luer housing is formed integral to the hub.

17. The catheter of claim 16 further comprising:
the elongate flexible shaft further comprising a second lumen and
a second luer, the second luer comprising:
a second proximal luer housing comprising a second proximal opening,
a second distal luer housing comprising a second stern and a second distal opening, and
a second threaded element disposed on an external surface of the second luer;
wherein the second proximal housing and the second distal housing define a second fluid channel,
wherein the second proximal housing is slidably attached to the second distal housing so that in a closed configuration the second proximal opening is fluidly sealed by the second stem, and in an open configuration the second proximal opening is in fluid communication with the second fluid channel and the second lumen, and
wherein the second distal luer housing is formed integral to the hub.

18. The catheter of claim 17, wherein at least one of the first distal luer housing, the first proximal luer housing, the second distal luer housing, and the second distal luer housing comprises a translucent material.

19. The catheter of claim 17, wherein at least one of the first distal luer housing and the first proximal luer housing comprise a first color indicator, the second distal luer housing and the second distal luer housing comprise a second color indicator, and wherein the first color indicator is different than the second color indicator.

20. The catheter of claim 17, wherein the hub comprises a translucent material.
